(19) United States
(12) Patent Application Publication
(10) Pub. No.: US 2002/0165592 A1
(43) Pub. Date: Nov. 7, 2002

(54) Induction powered in vivo imaging device

(76) Inventors: Arkady Glukhovsky, Nesher (IL); Gavriel J. Iddan, Haifa (IL); Gavriel Meron, Petach Tikva (IL)

Correspondence Address: Eitan, Pearl, Latzer & Cohen-Zedek, One Crystal Park, Suite 210, 2011 Crystal Drive, Arlington, VA, 22202-3709, US

(21) Appl. No.: 10/115,585

(22) Filed: Apr. 4, 2002

Related U.S. Application Data
(60) Provisional application No. 60/281,013, filed on Apr. 4, 2001.

(51) Int. Cl. A61N 1/08

(52) U.S. Cl. 607/62

(57) ABSTRACT

An in vivo imaging device including at least one image sensor and an energy receiving unit that is configured to receive electromagnetic energy and to convert the received electromagnetic energy to energy for powering at least one electrical component of the image sensor.

### PRIOR PROVISIONAL

The present application clams benefit from U.S. Provisional Application No. 60/281,013, filed on Apr. 4, 2001, entitled “Induction Powered In Vivo Imaging Device”.

### FIELD OF THE INVENTION

The present invention relates to induction powered in vivo devices, specifically to externally powered in vivo imaging devices.

### BACKGROUND OF THE INVENTION

Physiological sensors and medical devices such as cochlear prosthesis, artificial hearts and defibrillators may be implanted for performing in vivo. The implanted devices may contain a battery or may be powered externally. External energy transmission to implants increases the power efficiency and operative hours of the implanted devices. External wireless energy transmission to implants also contributes to patient motility and to the elimination of the potential of infection.

Transcutaneous coupling of power to implanted devices is one alternative for external energy transmission. Another alternative is described in WO 98/29030; which relates to an implantable stent for measuring fluid flow that call be powered by electrical energy received from a source out side the body. The stent circuitry can be activated by a time varying magnetic field that is generated in the vicinity of the stent coil and that is generally aligned with the central axis of the stent.

### SUMMARY OF THE INVENTION

The present invention provides an induction powered in vivo imaging device. The imaging device according to an embodiment of the invention may be moved through body lumens and thus may have an inconstant axis orientation,

The imaging device, according to an embodiment of the invention, includes an image sensors optionally an illumination source and an energy receiving unit. The energy receiving unit is configured for receiving electromagnetic energy and for converting the received electromagnetic energy to energy for powering at least one electrical component of the image sensor.

As referred to herein the term “electromagnetic energy” may refer to energy generated by an electromagnetic wave or by a magnetic field.

For example, the energy receiving unit may include at least one coil configured to receive electromagnetic energy and an element, coupled to the coil, configured for converting the received electromagnetic energy to energy for powering the electrical components of the device, such as the image sensors illumination source etc. The energy receiving unit may further be configured for storing the voltage, such as by including a capacitor or chargeable battery.

According to an embodiment of the invention, the imaging device images in vivo sites that are illuminated by the illumination source. The images may be stored in the imaging device or may be transmitted to an external receiving system. Thus, the device of the invention may further include a storing device, such as a solid state memory chip for the collected images. Alternatively, the device may include a transmitter for transmitting signals to an external receiving system.

Also provided, according to an embodiment of the invention, is a system for induction powered in vivo imaging. The system, according to an embodiment of the invention, includes an in vivo imaging device and an external energy source for induction of the imaging device, In one embodiment of the invention the in vivo imaging device contains an image sensor, an illumination source and an energy receiving unit. The device may further include a transmitter for transmitting signals to an external receiving system.

The external energy source for induction of the imaging device is typically a magnetic field generator capable of generating a time varying magnetic field around the in vivo imaging device. The varying magnetic field can be generated by an AC induction coil or by a rotating magnetic circuit,

The magnetic field generator may be in communication with or may include a localizing device for localizing the in vivo imaging device in a patient's body. The magnetic filed generator can then be moved along the patient's body in accordance with the in vivo imaging device location, as determined by the localizing device, thus optimizing the energy transfer from the external energy source to the in vivo imaging device.

In an embodiment of the invention the in vivo imaging device contains at least one complementary metal oxide semiconductor (CMOS) imaging camera, at least one light emitting diode (LED) and a transmitter for transiting video signals from the CMOS imaging camera to an external receiving system. The energy receiving unit contains a three axial coil assembly and a corresponding selector rectifier circuit that is able to convert magnetically induced AC voltage to a desired DC voltage that is available for powering the electrical components of the in vivo imaging device. The external energy source is a magnetic field generator containing a low frequency AC induction coil or a rotating magnetic circuit.

In another embodiment the energy receiving unit contains a single coil and the external energy source is a magnetic field generator having three alternating orthogonal components.

The magnetic field generator may be in communication with or may include a localizing device for localizing the in vivo imaging device in a body lumen. The magnetic filed generator can then be moved along a patient's body in accordance with the in vivo imaging device location, as determined by the localizing device for optimizing the energy transfer from the external energy source to the in vivo imaging device.

The device and system of the invention can be used for imaging body lumens, such as the gastrointestinal (GI) tract. The device according to an embodiment of the invention may be contained within a swallowable capsule that is capable of passing through and obtaining images of substantially the entire GI tract. Optionally, the device of the invention may be attached onto any device suitable for being inserted into and moved through body lumens, such as needles, stents, catheters and endoscopes.

Further provided according to an embodiment of the invention is a method for in vivo imaging. In one embodiment the method includes the step of: externally powering an in vivo imaging device to obtain images in vivo, the in vivo imaging device including at least one image sensor, optionally an illumination source and an energy receiving unit. The energy receiving unit is configured for receiving electromagnetic energy and for converting the received electromagnetic energy to energy for powering at least one electrical component of the image sensor.

Externally powering the in vivo imaging device can be done by generating a magnetic field around the in vivo imaging device. The magnetic field, which, according to some embodiments, may be unidirectional or having three orthogonal components, is generated around an area of the patient's body that contains the in vivo imaging device.

The method, according to an embodiment of the invention may further include the step of localizing the in vivo imaging device, optionally, prior to the step of externally powering the in vivo imaging device and the step of moving an external energy source, for example, a magnetic field generator, to correlate with the location of the in vivo imaging device in the patient's body.

In an embodiment of the invention the method may be useful for imaging the GI tract and the in vivo imaging device may be contained in a swallowable capsule that can pass through substantially the entire GI tract.

### BRIEF DESCRIPTION OF THE DRAWINGS

The present invention will be understood and appreciated more fully from the following detailed description taken in conjunction with the drawings in which:

FIG. 1A is a schematic illustration of the in vivo imaging device in accordance with an embodiment of the invention;

FIG. 1B is an electric block diagram of the energy receiving unit included in the in vivo imaging device illustrated in FIG. 1A, according to an embodiment of the invention;

FIG. 2A is a schematic illustration of the energy receiving unit in accordance with an embodiment of the invention;

FIG. 2B is an electric block diagram of the energy receiving unit illustrated in FIG. 2A, according to an embodiment of the invention;

FIG. 3 is a schematic illustration of the energy receiving unit in accordance with another embodiment of the invention; and

FIG. 4 is a schematic illustration of the system in accordance with an embodiment of the invention.

### DETAILED DESCRIPTION OF THE INVENTION

The device according to an embodiment of the present invention is an induction powered in vivo imaging device. The device may be introduced into a patient's body and the electrical components of the device may be powered by an external energy source that is applied to the patient's body. Thus, the device may not be dependent for its operation on a battery having a limited shelf life and a limited amount of operational hours.

The in vivo imaging device can be used to obtain images from within body lumens, inter alia, by being moved through the body lumen. The device can be attached onto a medical instrument designed to be inserted and/or moved in a body lumen, such as a swallowable capsule, needle, stent, catheter, endoscope, etc.

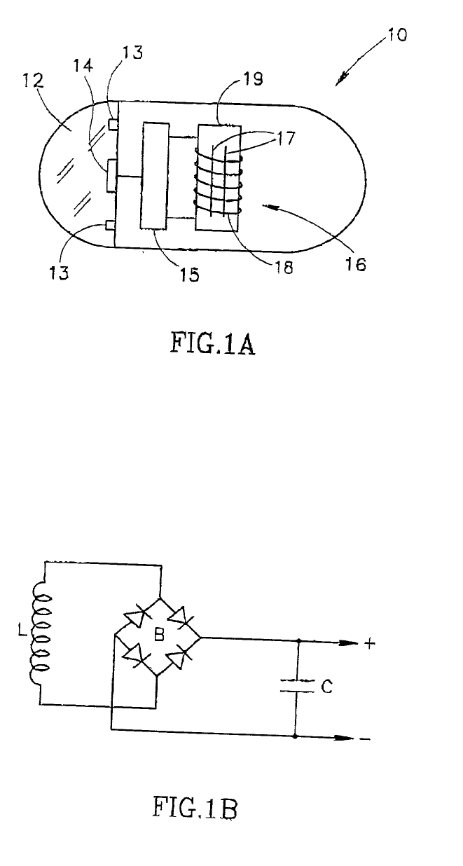

In an embodiment of the invention illustrated in FIG. 1, the device is contained within a swallowable capsule, such as the capsule described in WO 01/65995, which is assigned to the common assignee of the present invention and which is hereby incorporated by reference in its entirety.

The swallowable capsule 10 consists of an optical window 12 behind which are positioned at least one solid state imaging chip such as a CCD or CMOS imaging camera 14 for obtaining images of the GI tract and at least one LED 13 for illuninating the GI tract. The CMOS imaging camera 14 is connected to a transmitter 15 that transmits the video signals obtained by the CMOS imaging camera 14 to an external receiving system (not shown). The transmitter 15, CMOS imaging camera 14 and LEDs 13 are all connected to and powered by energy receiving unit 16.

Energy receiving unit 16 consists of an element 18, for example a conductive coil, configured for receiving energy from an external energy source, a rectifier circuit 19 for converting AC voltage to DC voltage and a capacitor 17. A capacitor ranging from several mili-Farads to a few hundred mili-Farads may be used or alternatively, a chargeable battery could be used for storage of the voltage required for operation of the electrical components of the capsule 10. For example, a capacitor of about 10 Farad and 5 mWatt is suitable for use in the present invention.

A block diagram of the energy receiving unit 16 is illustrated in FIG. 1B. A single receiving inductor L converts a time varying magnetic field into alternating electrical current which is rectified by diode bridge B. Capacitor C serves as an energy storing and ripple damping element.

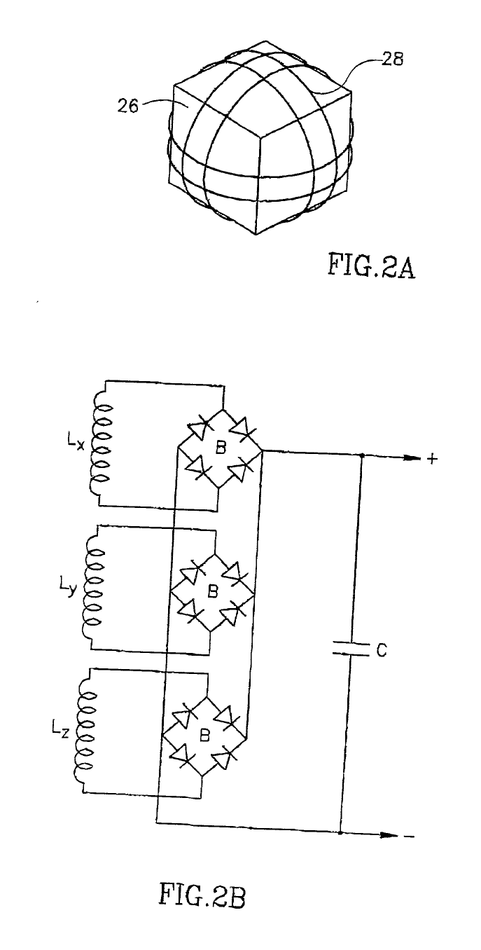

Reference is now made to FIG. 2A which is a schematic illustration of one embodiment of the energy receiving unit of the invention. Energy receiving unit 26 includes a capacitor or any other suitable energy storing component (not shown), a to rectifier circuit for converting AC voltage to DC voltage (not shown) and a three axial coil assembly 28 or possibly, three or more separate orthogonal elements configured for receiving energy from an external energy source. The three axial coil assembly 28 ensures that energy will be produced from a unidirectional magnetic field independently of the directionality of the energy receiving unit 26 (as will be further discussed below).

A block diagram of the energy receiving unit 26 is illustrated in FIG. 2B, Three orthogonal coils Lx, Ly and Lz convert a time varying magnetic field into alternating electrical current which is rectified by diode bridges B. Capacitor C serves as an energy storing and ripple damping element.

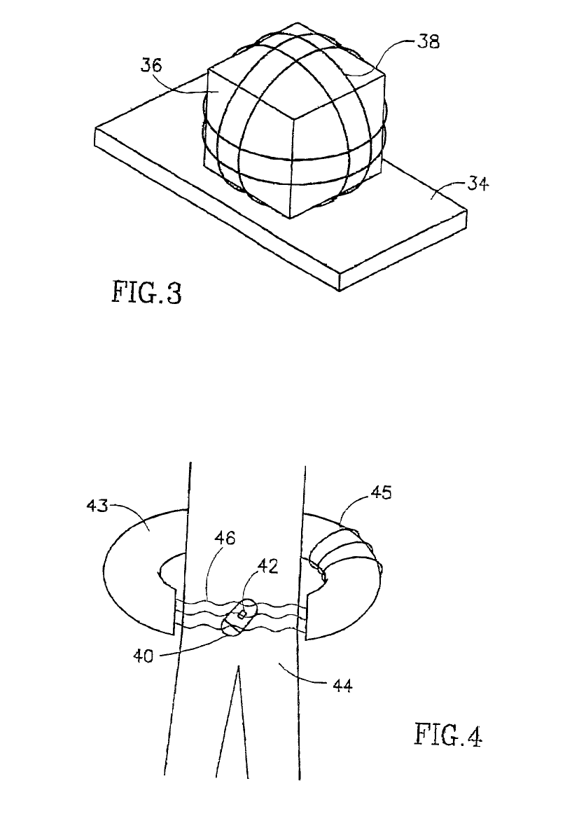

Another embodiment of the energy receiving unit of the invention is schematically presented in FIG. 3. Energy receiving unit 36, which includes a three axial coil assembly 38, a capacitor (not shown) and a rectifier circuit for converting AC voltage to DC voltage (not shown), is connected to a circuit 34 capable of selecting the coil having the maximal voltage, rectifying and stabilizing it to a desired voltage by methods known in the art. Energy transfer to a device which includes energy receiving unit 36, coil assembly 38 and circuit 34 is thus optimized.

Reference is now made to FIG. 4 in which an embodiment of the system of the invention is schematically illustrated. A medical instrument 40 containing the device 42 of the invention, such as the capsule described in FIG. 1, is introduced into a patient's body 44. A varying magnetic field 46 is generated by magnetic field generator 43 around the patient's body 44, in the area containing the medical instrument 40. Magnetic field generator 43 can include an AC indunction coil 45, typically a low frequency Ac indunction coil (about 60 Hz) or may have a rotating magnetic circuit to generate a varying magnetic field. In order to achieve higher efficiency of the energy transmission it may be desirable to operate in a relatively high frequency range. However, due to high attenuation of the body tissues at high frequencies—the practical frequency range will typically be from several tens of Hz to several tens of KHz.

The magnetic field 46 is received by an element configured for receiving energy in device 42. The magnetic field 46 induces a current in the element which can be received (and stored) by a capacitor for powering the electrical components of the medical instrument 40.

Magnetic field 46 may be generated by three orthogonal coils surrounding the patient's body 44. This configuration enables the receiving element in device 42 to have an arbitrary orientation in the patient's body 44 and yet to be able to pick up energy from the generated magnetic field 46,

In generating a magnetic field around the patient's body 44 three orthogonal external coils may be operated simultaneously, with the same phase, adding up to a linear magnetic field. Alternatively, the coils may be operated either sequentially or with a phase shift between them, resulting in a magnetic field with time-varying orientation.

Induction of an electromagnetic field in the receiving element may be most efficient when the long axis of the receiving element and the magnetic field 46 axis are orthogonal to each other. However, since medical instrument 40 may move through a body lumen, sometimes rotating or tumbling through the lumen, the directionality of the device 42 (and of the element in it) is not always permanent and not necessarily known, making it difficult to keep the magnetic field and the axis of the element in a fixed position relative to each other. This problem is overcome in the present invention in one of two ways; the element configured for receiving energy comprises a three axial coil array or the magnetic field includes a three axial arrangement, such that for any directionality of the medical instrument 40, and of the device 42 in it, there is a magnetic field basically orthogonal to the long axis of the energy receiving element.

In an embodiment of the invention the location of the medical instrument 40, at any given moment, can be determined and the magnetic field generator 43 can be moved to correlate with the location of the medical instrument 40 in the patient's body 44. I this embodiment the system may include a reception system located externally, typically comprising an antenna array wrapped around the central portion of the patient's trunk; other reception systems are possible. The antennas are located so as to be able to determine from their output the location of the medical instrument 40 within the patient's body 44. The output of the antennas can be, used to determine the location of the medical instrument 40 by triangulation or any other suitable method known in the art. For example, a method for determining the location of a swallowable capsule in a patient's GI tract is described in U.S. Pat. No. 5,604,531. U.S. Pat. No. 5,604,531, which is assigned to the common assignee of the present application, is hereby incorporated by reference in its entirety.

The determined location of medical instrument 40 can be displayed two- or three-dimensionally on a position monitor, typically, though not necessarily, as an overlay to a drawing of the body lumen it is in, such as the digestive tract.

The magnetic field generator 43 may be in communication with the position monitor such that the location of the magnetic field generator 43 can be correlated to that of the medical instrument 40 in the patient's body 44. Alternatively, the magnetic field generator 43 may include a localizing device for localizing the medical device 40 in the patient's body 44, in a similar manner to that described above, The magnetic filed generator 43 can then be moved along the patient's body 44 in accordance with the location of the medical device 40, thus optimizing the energy transfer from the external energy source to the medical instrument 40.

It will be appreciated by persons skilled in the art that the present invention is not limited to what has been particularly shown and described hereinabove. Rather the scope of the present invention is defined only by the claims which follow:

1. An in vivo imaging device comprising at least one image sensor; and
an energy receiving unit configured to receive electromagnetic
energy and to convert the received electromagnetic energy to energy for powering at least one electrical component of the image sensor.

2. The imaging device according to claim 1 further comprising an illumination source.

3. The imaging device according to claim 2 wherein the illumination source is positioned behind an optical window.

4. The imaging device according to claim 2 wherein the image sensor and the illumination source are positioned behind an optical window.

5. The imaging device according to claim 2 wherein the illumination source is an LED.

6. The imaging device according to claim 1 wherein the energy receiving unit comprises at least one coil configured to receive electromagnetic energy.

7. The imaging device according to claim 1 wherein the energy receiving unit comprises three coils configured to receive electromagnetic energy.

8. The imaging device according to claim 1 wherein the energy receiving Unit comprises three orthogonal coils configured to receive electromagnetic energy.

9. The imaging device according to claim 1 wherein the energy receiving unit is configured to produce energy from a magnetic field independently of the directionality of the energy receiving unit.

10. The imaging device according to claim 9 wherein the energy receiving unit comprises three orthogonal coils configured to receive electromagnetic energy.

11. The imaging device according to claim 1 wherein the energy receiving unit comprises a rectifier circuit.

12.The imaging device according to claim 1 wherein the energy receiving unit is configured to store energy.

13. The imaging device according to claim 1 wherein the energy receiving unit comprises a capacitor or a chargeable battery.

14.The imaging device according to claim 1 wherein the image sensor is a CCD or a CMOS imaging camera.

15. The imaging device according to claim 1 further comprising a transmitter.

16. The imaging device according to claim 1 further comprising a transmitter for transmitting image data.

17.An in vivo imaging device comprising
at least one image sensor configured to obtain video signals; and
an energy receiving unit configured to receive electromagnetic energy and to convert the received electromagnetic energy to energy for powering at least one electrical component of the image sensor.

18. A system for in vivo imaging, said system comprising
an in vivo imaging device and an external energy source configured to induce the imaging device,
said in vivo imaging device comprising at least one image sensor; and
an energy receiving unit configured to receive electromagnetic energy and to convert the received electromagnetic energy to energy for powering at least one electrical component of the image sensor.

19. The system according to claim 18 wherein the external energy source generates a time varying magnetic field.

20. The system according to claim 18 wherein the external energy source is a magnetic field generator.

21. The system according to claim 20 wherein the magnetic field generator comprises three alternating orthogonal components.

22. The system according to claim 18 further comprising a localizing device configured to localize the in vivo imaging device in a body lumen.

23. A method for in vivo imaging, the method comprising the step of externally powering an in vivo imaging device according to claim 1 to obtain images in vivo.

24. A method for in vivo imaging, the method comprising the step of externally powering an in vivo video imaging device to obtain images in vivo.

25. The method according to claim 23 further comprising the step of transmitting image signals to an external receiving unit.

26. The method according to claim 24 further comprising the step of transmitting video signals to an external receiving unit.

27. The method according to claim 23 further comprising the steps of
localizing the in vivo imaging device; and
moving an external energy source to correlate with the location of the in vivo imaging device.

28. The method according to claim 24 further comprising the steps of
localizing the in vivo imaging device; and
moving an external energy source to correlate with the location of the in vivo imaging device.

29. A capsule for imaging the gastrointestinal tract, the capsule comprising
at least one image sensor, and
an energy receiving unit configured to receive electromagnetic energy and to convert the received electromagnetic energy to energy for powering at least one electrical component of the image sensor.

30. A capsule for imaging the gastrointestinal tract, the capsule comprising
at least one CMOS image sensor;
at least one LED;
a transmitter for transmitting; image signals to an external receiving unit; and
an energy receiving unit configured to receive electromagnetic energy and to convert tie received electromagnetic energy to energy for powering at least one electrical component of the image sensor.
